(19) United States
(12) Patent Application Publication
(10) Pub. No.: US 2024/0176128 A1
(43) Pub. Date: May 30, 2024

(54) MAGNETO-PHOTONIC PARTICLES

(71) Applicant: Regents of the University of Minnesota, Minneapolis, MN (US)

(72) Inventors: Ognjen Ilic, Roseville, MN (US); Seung Yeol Lee, Minneapolis, MN (US)

(21) Appl. No.: 18/520,008

(22) Filed: Nov. 27, 2023

Related U.S. Application Data
(60) Provisional application No. 63/428,231, filed on Nov. 28, 2022.

(51) Int. Cl. G02B 26/00; G21K 1/093

(52) U.S. Cl. CPC G02B 26/00; G21K 1/093

(57) ABSTRACT

Methods, devices, and systems for controllable magneto-photonic particles (e.g., for light delivery and/or collection) are provided. In one aspect, a magneto-photonic particle includes a supporting material, a photonic structure configured to manipulate light, and a magnetic structure controllable to make the particle move. The photonic structure and the magnetic structure are supported by the supporting material.

### CROSS-REFERENCE TO RELATED APPLICATIONS

This application claims the benefit of U.S. Patent Application Ser. No. 63/428,231, filed on Nov. 28, 2022. The disclosure of the prior application is considered part of (and is incorporated by reference in) the disclosure of this application.

### TECHNICAL FIELD

This present disclosure is related to controllable signal delivery and/or collection.

### BACKGROUND

The ability to deliver and collect light to and from a target spot in a medium has broad relevance for applications across biology and biomedicine, from neural stimulation and imaging to drug delivery and thermotherapy.

### SUMMARY

The present disclosure provides methods, devices, systems for controllable magneto-photonic particles, e.g., for light collection and delivery.

One aspect of the present disclosure features a particle including: a supporting material; a photonic structure configured to manipulate light; and a magnetic structure controllable to make the particle move, where the photonic structure and the magnetic structure are supported by the supporting material.

In some embodiments, the photonic structure is configured to diffract, steer, reflect, focus, scatter, direct, absorb, or transmit the light.

In some embodiments, the photonic structure is configured to deliver the light towards a target in a medium, to focus the light from the target in the medium, to collect the light from a target in a medium, to diffract light with a first wavelength towards a first direction and light with a second wavelength towards a second direction, to diffract light with a first wavelength and absorb light with a second wavelength, or a combination thereof.

In some embodiments, the photonic structure includes at least one of a micro-photonic structure, nano-photonic structure, one-dimensional (1D) structure, two-dimensional (2D) structure, three-dimensional (3D) structure, diffractive grating, or a combination thereof.

In some embodiments, the magnetic structure is configured to be directionally orientable, has a non-circularly symmetric shape, has a triangular shape, includes a solid structure made of a magnetic material, includes an engineered structure of a magnetic material, the engineered structure configured to transmit the light from the photonic structure, includes cobalt, nickel, iron, iron-oxide, an alloy comprising at least one magnetic material, or a combination thereof.

In some embodiments, the supporting material includes at least one of a polymer material, a dielectric material, a semiconducting material, a non-magnetic metal, or a photoresist material.

In some embodiments, the particle has a disc shape with a diameter at an order of ones to tens of micrometers.

In some embodiments, the photonic structure is formed on top of the supporting material.

In some embodiments, the magnetic structure is below the photonic structure and embedded in the supporting material.

In some embodiments, the magnetic structure is on a bottom of the supporting material.

In some embodiments, the supporting material includes a top surface and a bottom surface along a direction. The photonic structure can be formed on the top surface of the supporting material, and the magnetic structure can be embedded in the supporting material

In some embodiments, a thickness of the supporting material from the top surface to the bottom surface along the direction is in an order of micrometers.

In some embodiments, the magnetic structure has an upper edge and lower edge, and has a thickness from the upper edge to the lower edge along the direction in an order of tens of nanometers.

In some embodiments, a distance from the top surface to the upper edge of the magnetic structure is substantially identical to a distance from the lower edge of the magnetic structure to the bottom surface.

In some embodiments, a distance from the top surface to the upper edge of the magnetic structure is larger than a distance from the lower edge of the magnetic structure to the bottom surface.

In some embodiments, the upper edge and lower edge of the magnetic structure each have a substantially isosceles triangular shape in a plane perpendicular to the direction. A width and a height of the triangular shape can be at an order of tens of micrometers.

In some embodiments, an outer surface of the particle includes one or more functional chemical or biological groups.

Another aspect of the present disclosure features a particle including: a supporting material; a photonic structure configured to manipulate an electromagnetic signal; and a magnetic structure controllable to make the particle move, where the photonic structure and the magnetic structure are supported by the supporting material.

In some embodiments, the electromagnetic signal includes at least one of ultraviolet light, visible light, infrared light, a radio-frequency (RF) signal, a microwave signal, or an acoustic signal.

In some embodiments, the supporting material includes at least one of a polymer material, a dielectric material, a semiconducting material, a non-magnetic metal, or a photoresist material.

In some embodiments, the magnetic structure comprises at least one of cobalt, nickel, iron, iron-oxide, or an alloy including at least one magnetic material.

In some embodiments, the photonic structure is configured to manipulate the electromagnetic signal by diffracting, steering, reflecting, focusing, scattering, directing, absorbing, or transmitting the electromagnetic signal.

Another aspect of the present disclosure features a method of forming magneto-photonic particles including: forming multiple discrete supporters on a substrate; forming respective magnetic structures on the multiple discrete supporters; forming a respective photonic structure above each of the respective magnetic structures; and releasing the multiple discrete supporters from the substrate to form multiple magneto-photonic particles each having a photonic structure and a magnetic structure.

In some embodiments, forming the multiple discrete supporters includes: depositing a supporting material on the substrate; and patterning the supporting material according to a pattern corresponding to the multiple discrete supporters on the substrate.

In some embodiments, forming the respective magnetic structures on the multiple discrete supporters includes: depositing a magnetic layer on the multiple discrete supporters; and performing, on each discrete supporter, a lift-off of a portion of the magnetic layer. The magnetic layer can include at least one of cobalt, nickel, iron, iron-oxide, or an alloy including at least one magnetic material.

In some embodiments, the method includes forming a separation layer on top of the respective magnetic structures.

In some embodiments, the multiple discrete supporters and the separation layer include a same material.

In some embodiments, the multiple discrete supporters include a first material, and the separation layer include a second, different material.

In some embodiments, the respective magnetic structures include a magnetic material compatible with a material of the multiple discrete supporters.

In some embodiments, forming the respective photonic structure on the separation layer and above each of the respective magnetic structures includes: imprinting the separation layer with a photonic pattern on a stamp or patterning the photonic structure using e-beam lithography.

In some embodiments, forming the respective photonic structure on the separation layer and above each of the respective magnetic structures includes: depositing a metallic layer on the imprinted pattern or patterning a metallic layer using e-beam lithography; or both. The metallic layer can include a metal.

In some embodiments, releasing the multiple discrete supporters from the substrate to form the multiple magneto-photonic particles includes: dissolving the substrate to release the multiple discrete supporters.

In some embodiments, each of the magneto-photonic particles includes the particle as described in the present disclosure.

Another aspect of the present disclosure features a method of operating magneto-photonic particles including: illuminating, with an electromagnetic (EM) signal, a magneto-photonic particle in a medium; and moving the magneto-photonic particle to a vicinity of a target in the medium by controlling a magnetic structure in the magneto-photonic particle, such that a photonic structure of the magneto-photonic particle delivers the EM signal to the target.

In some embodiments, the method further includes orienting the magneto-photonic particle to direct the EM signal by the photonic structure to the target, collecting another EM signal coming from the target by the photonic structure of the magneto-photonic particle, or both.

In some embodiments, the EM signal coming from the target includes at least one of: a scattered EM signal, a reflected EM signal, or an emitted EM signal.

Another aspect of the present disclosure features a method of operating magneto-photonic particles including: moving a magneto-photonic particle to a vicinity of a target in a medium by controlling a magnetic structure in the magneto-photonic particle; and collecting an electromagnetic (EM) signal from the target by a photonic structure of the magneto-photonic particle.

Another aspect of the present disclosure features a method of operating magneto-photonic particles including: moving a magneto-photonic particle to a vicinity of a target in a medium by controlling a magnetic structure in the magneto-photonic particle; and illuminating, with an electromagnetic (EM) signal, the magneto-photonic particle to deliver the EM signal by a photonic structure of the magneto-photonic particle to the target.

In some embodiments, moving the magneto-photonic particle to the vicinity of the target in the medium includes: translating or rotating a magnetic controller relative to the magneto-photonic particle.

In some embodiments, translating or rotating the magnetic controller relative to the magneto-photonic particle causes the magneto-photonic particle to translate or rotate relative to the magnet.

Another aspect of the present disclosure features a system including: one or more magneto-photonic particles each according to the particle as described in the present disclosure; and a magnetic controller configured to directionally move the one or more magneto-photonic particles.

In some embodiments, the system further includes: an electromagnetic (EM) source configured to illuminate an EM signal on the one or more magneto-photonic particles, an EM signal detector configured to detect an EM signal from a target in a vicinity of the one or more magneto-photonic particles, or both.

The details of one or more embodiments of the subject matter described in the present disclosure are set forth in the accompanying drawings and description below. Other features, aspects, and advantages of the subject matter will become apparent from the description, the drawings, and the claims.

### BRIEF DESCRIPTION OF THE DRAWINGS

FIG. 1 is a schematic diagram of an example system using magneto-photonic particles.

FIG. 2A illustrates various examples of magneto-photonic particles for light delivery and collection with corresponding photonic structures.

FIG. 2B illustrates examples of magneto-photonic particles for light manipulation with corresponding wavelength dependent photonic structures.

FIG. 3 illustrates an example magnetic structure in a magneto-photonic particle.

FIG. 4 shows examples of controllable movements of a magneto-photonic particle with a non-symmetric magnetic structure.

FIG. 5 shows an example process of fabricating magneto-photonic particles.

FIG. 6A shows an example fabricated magneto-photonic particle.

FIG. 6B shows an example system for controlling the magneto-photonic particle of FIG. 6A.

FIG. 6C shows results of controlling light steering directions of the magneto-photonic particle of FIG. 6A by controlling orientations of the magneto-photonic particle.

FIG. 7 is a flowchart of an example process for forming magneto-photonic particles.

FIG. 8A is a flow chart of an example process for operating magneto-photonic particles for delivering light.

FIG. 8B is a flow chart of another example process for operating magneto-photonic particles for delivering light.

FIG. 8C is a flow chart of another example process for operating magneto-photonic particles for collecting light.

Like reference numbers and designations in the various drawings indicate like elements. It is also to be understood that the various example implementations shown in the figures are merely illustrative representations and are not necessarily drawn to scale.

### DETAILED DESCRIPTION

Implementations of the present disclosure provide controllable magneto-photonic particles, e.g., for light collection and/or delivery where the particles can be remotely actuated with a magnetic field. The magneto-photonic particles can include an optically active surface with a magnetic core. Integrating a photonic structure on the surface of a magneto-photonic particle allows for complex optical responses, and the magnetic core facilitates controlled steering and movement of the magneto-photonic particle. The design parameters of the magneto-photonic particles can be chosen to allow both optical and magnetic functionalities.

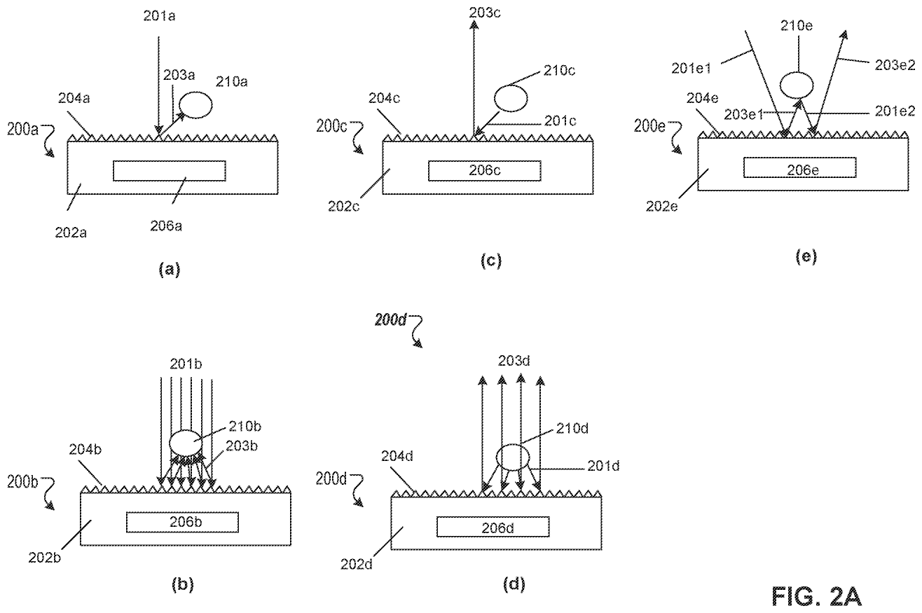

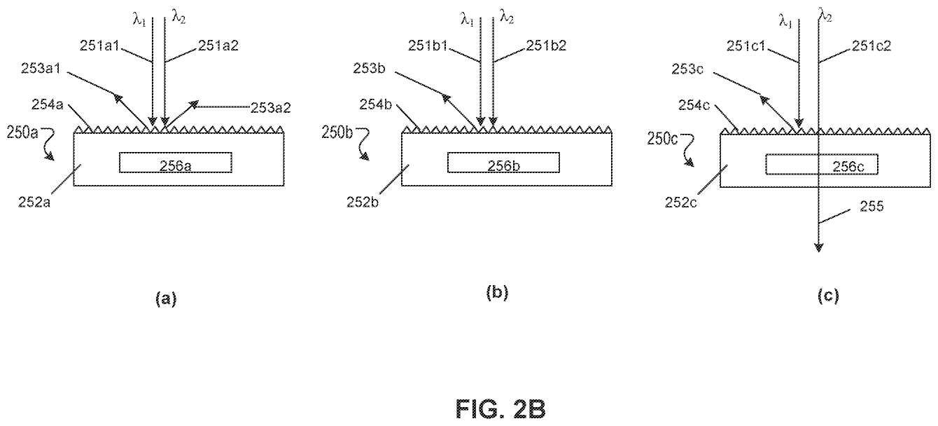

In some embodiments, e.g., as illustrated with further details in FIGS. 2A-2B, the photonic structure of the magneto-photonic particle can be configured for light manipulation, e.g., optical functionalities such as diffraction, scattering, reflection, absorption, transmission, and wavelength dependence of those functionalities. The photonic structure can include subwavelength features to provide control of direction of scattering properties, e.g., by controlling the surface refractive index variation.

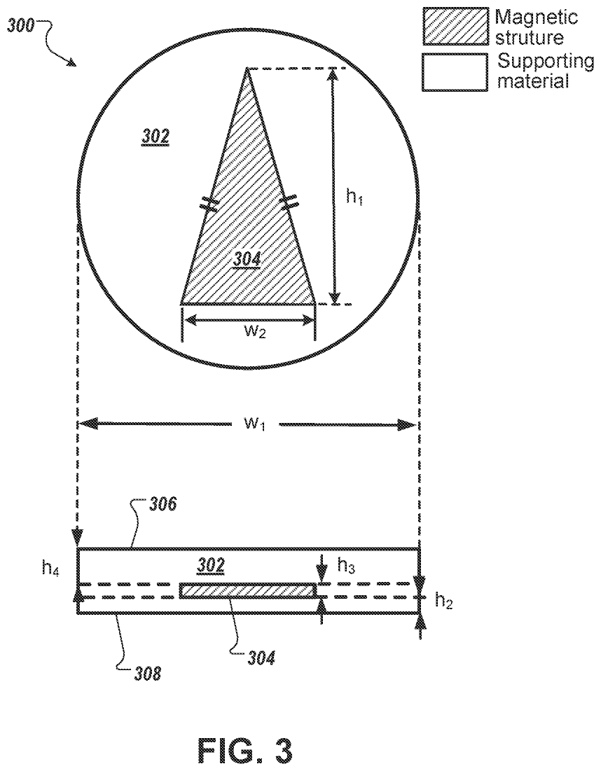

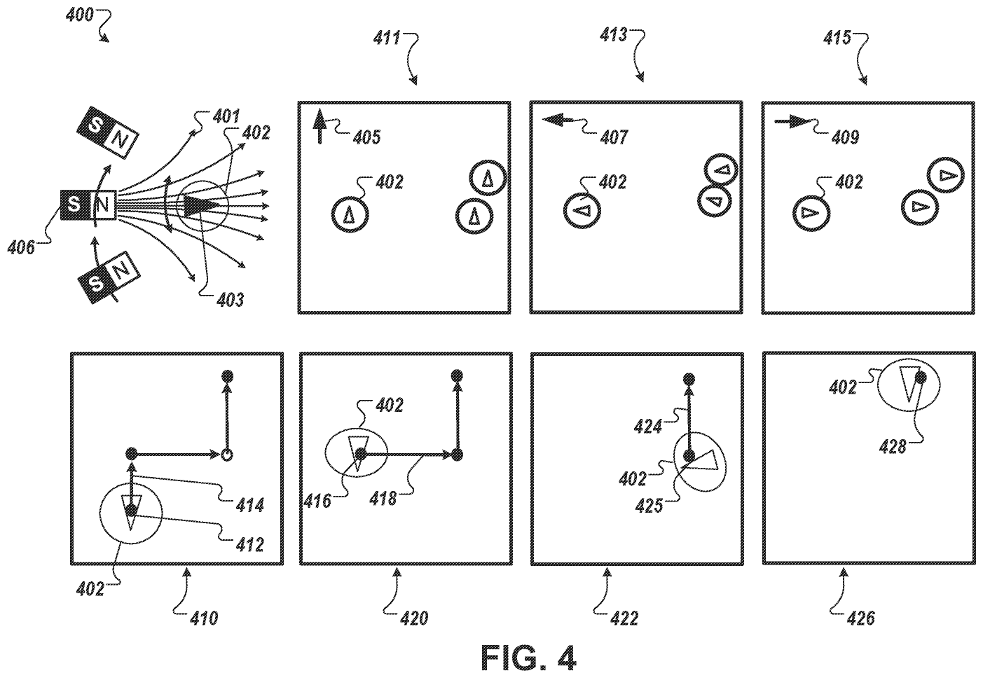

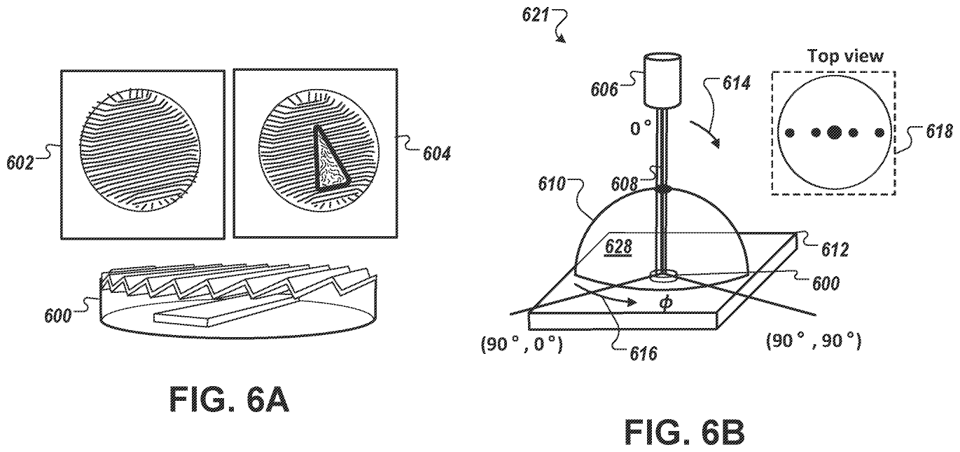

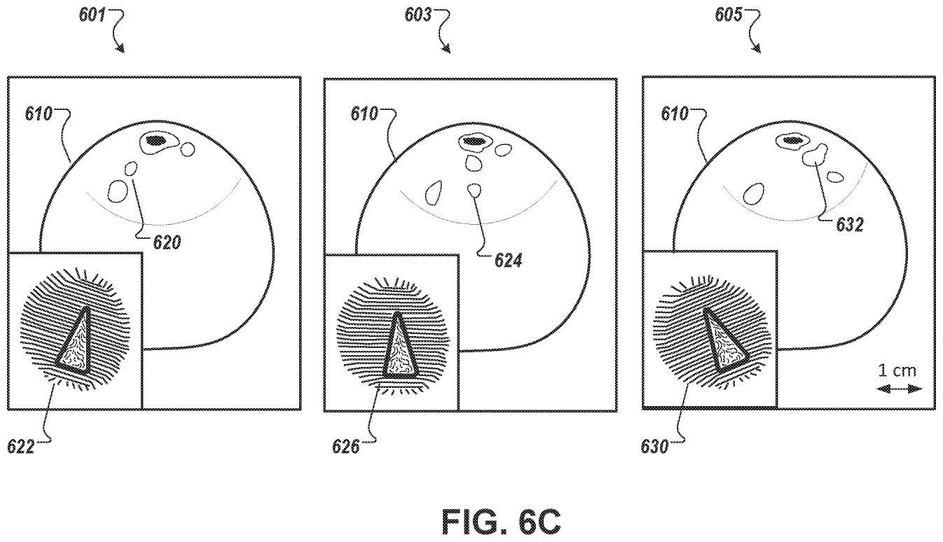

In some embodiments, e.g., as illustrated with further details in FIGS. 3 and 4, the magnetic structure in the magneto-photonic particle can be configured to be directionally orientable. For example, the magnetic structure can have a non-circularly symmetric shape, e.g., a triangle shape. In such a way, the magnetic structure can facilitate controlled steering and movement of the magneto-photonic particle, e.g., by a magnetic field. Further, the controllable orientation of the magneto-photonic particle enables directional control of light manipulated by the photonic structure, e.g., as illustrated with further details in FIGS. 6A-6C.

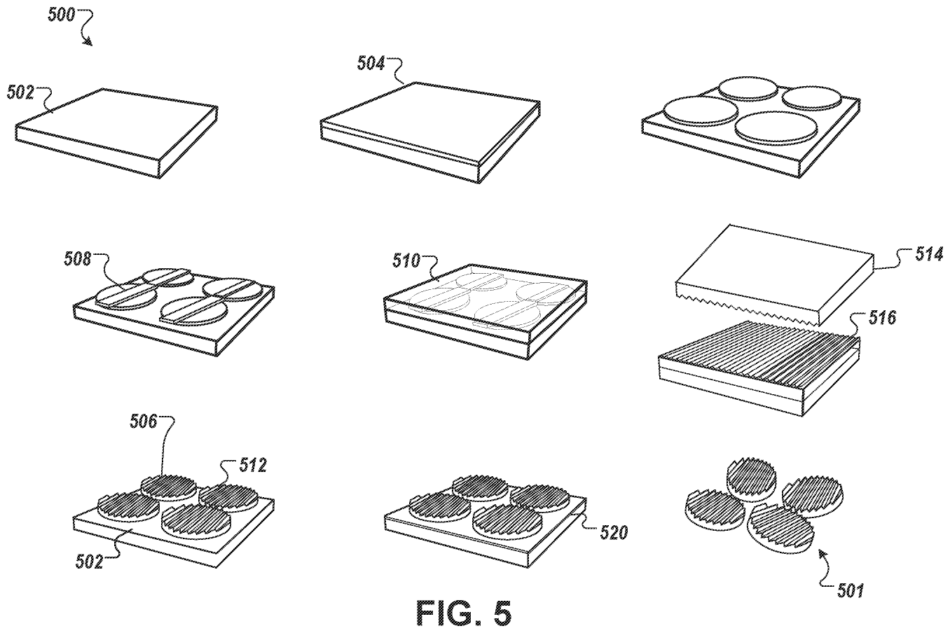

The photonic surface that provides sophisticated control of direction of scattering properties can be patterned onto the particle using nanoimprint lithography, e.g., as illustrated with further details in FIG. 5, which enables the production of complex features at nanoscale resolution, while being highly scalable. A surface photonic pattern of the photonic structure can be optimized or configured depending on its application.

Once the orientation of the pattern of a photonic surface of the photonic structure and direction of magnetization of the magnetic structure are synchronized, the position of a target spot of light delivery and/or collection can be controlled with an external magnetic field. Moreover, as the photonic structure is on a surface of the magneto-photonic structure, the surface of the photonic structure can be functionalized with functional chemical or biological groups, which can be used to bind targeted objects (e.g., molecules, cells, or tissues) for enhanced performance (e.g., stronger capability for light delivery and collection).

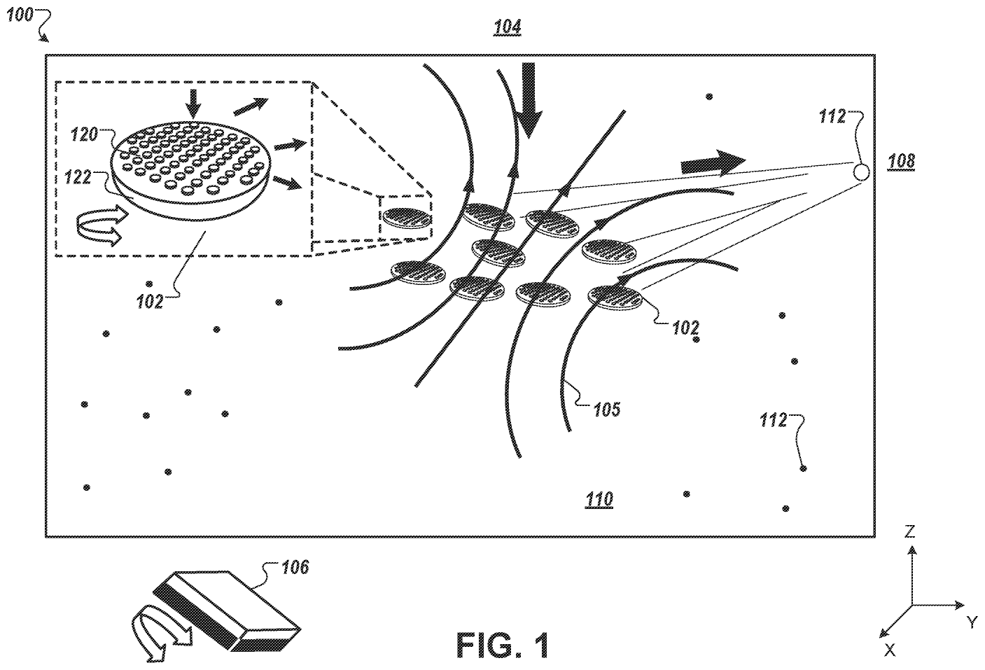

The combination of the photonic structure and the magnetic structure in a small size particle can result in an implantable and embeddable particle whose position and orientation can be externally controlled to deliver and collect light to a target (e.g., a molecule) in a medium, e.g., as illustrated with further details in FIG. 1.

The implementations described herein can provide various technical benefits and advantages. For example, the techniques enable magneto-photonic particles to deliver and/or collect light of desired properties (e.g., wavelength, intensity, polarization, etc.) to/from any target spot in a medium. The magneto-photonic particles can exhibit high directionality and significant selectivity for desired light properties, can be adaptively controlled with a non-invasive mechanism, and do not require direct line-of-sight access to the target spot. For example, the ability to translate and rotate a magneto-photonic particle within the medium i) facilitates light encountering the magneto-photonic particle when it would otherwise be difficult, e.g., the light source is fixed, and ii) enables light delivery and/or collection to a target when it would otherwise be difficult, e.g., when the target is fixed.

The techniques can provide a suitable platform that enables targeted delivery of light within relevant biological windows, with possible applications across biology and biomedicine fields, e.g., from opto-genetics, bio-electronics, neural stimulation and imaging to drug delivery and thermotherapy (such as non-invasive localized heating). For example, a magnet can externally control the particles when the particles are embedded within a medium (e.g., a biological or biomedical medium). The magneto-photonic particles can be used to send infrared light or localized heat to a tissue (e.g., for thermotherapy) or detect molecules, cells, or tissues (e.g., for sensing, neural stimulation). Also, the magneto-photonic particles can be used to improve imaging of biomaterials by increasing electromagnetic field strength and/or collecting light in a localized area (e.g., near-field imaging). Near-field imaging can be compromised when a camera is located external to the medium and outside of the diffraction limit for the object to be imaged. The magneto-photonic particles can enable imaging an object in the near field, e.g., the magneto-photonic particles and the object are within a few wavelengths of the light of each other.

Further, the magneto-photonic particles can be scalable in size, which can depend on applications, light wavelengths, particle materials, and/or targets. The size can be in a range, e.g., from 1 μm to 1 mm such as 10 μm to 100 μm or 50 μm to 100 μm. Compared to nanoparticles that operate via Rayleigh scattering, the size of the magneto-photonic particles allows for more control of the direction of light steering. The techniques enable large-volume and cost-effective fabrications of the magnetic-photonic particles, e.g., by nanoimprinting and semiconductor fabrication technologies. As different components (e.g., photonic structure and magnetic structure) in the magneto-photonic particles can be independent from each other to implement respective functionalities, different suitable combinations of materials can be chosen. The techniques can be applied to various types of signals, such as light (e.g., infrared light or visible light), other electromagnetic signals (e.g., radio-frequency signals, micro-wave signals), or acoustic signals (e.g., ultrasound signals). For example, photonic structures can be configured to have electromagnetic (EM) functionality at longer wavelengths than infrared light, e.g., radio-frequency signals or microwave signals, in which there may be biological transparency windows for biological or biomedical applications. For illustration purpose, light is used as an example of the EM signal in the present disclosure.

### Example Systems

FIG. 1 is a schematic diagram of an example system 100 using magneto-photonic particles. As illustrated in FIG. 1, the system 100 can include magneto-photonic particles 102 and a magnetic source 106. In some examples, the system 100 further includes at least one of a light source 104 or a light detector 108. The system 100 can be configured to control the magneto-photonic particles 102 to deliver light to and/or collect light from one or more targets 112 (e.g., molecules, cells, or tissues) in a medium 110 (e.g., a biological or biomedical medium).

The light source 104 is configured to provide light to illuminate the magneto-photonic particles 102. The light source 104 can include a laser, a light emitting diode (LED), or any suitable light source. In some examples, the light is an infrared light, e.g., with a wavelength in range from 780 nm to 1.4 μm or beyond. In some examples, the light is a visible light, e.g., with a wavelength in a range from 400 nm to 700 nm. In some examples, the light is an ultra-violet (UV) light, e.g., with a wavelength less than 400 nm. The light source 104 can be located externally relative to the medium 110, e.g., movable or fixable in location.

In some embodiments, a magneto-photonic particle 102 includes a magnetic structure. The magnetic structure can be embedded in the magneto-photonic particle 102 as a magnetic core. The magnetic source 106 can cause the magneto-photonic particles 102 to rotate, translate, or both. The magnetic source 106 can be a magnet, an electrical coil, or any suitable source that can generate a magnetic field. The magnetic source 106 generates a magnetic field 105, which interacts with the magnetic structure of the magneto-photonic particles 102. As an example, rotating the magnetic source 106 about the X axis can alter the orientation of the magnetic field 105 and cause the magneto-photonic particles 102 to rotate, thus changing their orientation, e.g., as illustrated with further details in FIG. 6C. As another example, the magnetic source 106 can be translated to a targeted spot along the Y axis or Z axis, e.g., as illustrated with further details in FIG. 4, increasing the strength of the magnetic field 105 experienced by the magneto-photonic particles 102. The magnetic source 106 can be externally located relative to the medium 110. In some embodiments, the magnetic source 106 is located within the medium 110 and an external magnetic controller is configured to control the magnetic source 106 to move.

In some embodiments, the magneto-photonic particle 102 includes a photonic structure 120. The photonic structure 120 can be formed on a surface of the magneto-photonic particle 102 and be configured for any suitable light manipulation, e.g., optical functionalities such as diffraction, scattering, reflection, absorption, transmission, and wavelength dependence of those functionalities. For example, the photonic structure 120 can include a photonic material that can include a dielectric material, e.g., having a dielectric constant k greater than one, a high refractive index material, e.g., having a refractive index great than 1.5, a non-magnetic material, or a combination thereof.

A surface photonic pattern of the photonic structure 120 can be optimized or configured depending on a particular application. Examples of the photonic structure 120 include, but are not limited to, diffraction gratings (e.g., transmissive or reflective), metastructures such as metagratings, and reflective mirrors. The photonic structure 120 can have wavelength dependence. For example, the spacing characterizing the photonic structure for infrared applications can be larger than the spacing for visible or ultraviolet applications. The photonic structure can include one-dimensional (1D) structure, two-dimensional (2D) structure, and/or three-dimensional (3D) structures. In some examples, e.g., as illustrated with further details in FIGS. 2A-2B, the photonic structure 120 can be configured to deliver light, to collect light, or to deliver and collect light. The photonic structure can be configured to act as or be a microlens or a focusing mirror to focus light to the target 112 or from the target 112.

As an example, as shown in FIG. 1, the photonic structure 120 of the magneto-photonic particle 102 is configured to deliver (e.g., steer) light to the target 112, e.g., by reflectively diffracting, steering, or reflecting light incident on the magneto-photonic particles 102. The steered light by the magneto-photonic particles 102 can be focused onto the target 112. Light from the target 112, e.g., scattered light, reflected light, or emitted light, can be collected or detected by the light detector 108. The inlet in FIG. 1 shows a close-up of an example magneto-photonic particle 102. The surface of the magneto-photonic particles 102 includes a 2D metagrating, e.g., a geometric pattern that creates varying index of refraction. The 2D metagrating can be configured to directionally steer the incident light.

As an example, FIG. 1 depicts light delivery, e.g., the magneto-photonic particles 102 redirect incident laser light from the light source 104 to the target 112. However, other methods involving the magneto-photonic particles 102 are possible. For example, a target 112 can emit light, e.g., under an excited light or without external excitation. The emitted light can be fluorescent light or luminescent light. The magneto-photonic particles 102 can be oriented and located to collect the emitted light, receive, and/or focus the emitted light at the target 112.

In some embodiments, the system 100 is configured to control a magneto-photonic particle 102 to collect light, deliver light, or both. In some embodiments, the system 100 is configured to control multiple magneto-photonic particles 102 acting as an ensemble to collectively collect light, deliver light, or both.

In some embodiments, as the photonic structure is on a surface of the magneto-photonic particle 102, the surface of the photonic structure can be functionalized (e.g., chemically or biologically) with functional groups (e.g., biological or chemical groups), which can be used to bind targeted objects (e.g., molecules, cells, or tissues) for enhanced performance (e.g., stronger light intensity for delivery and collection). For example, the magneto-photonic particles 102 can be chemically functionalized with functional groups to reduce a distance between the target 112 and the photonic structure 120, which can enhance light intensity and/or image quality of the target 112.

In some embodiments, the magneto-photonic particle 102 includes a supporting material 122 (as shown in the inlet of FIG. 1) that is configured to support the photonic structure 120 and the magnetic structure. The supporting material 122 can include any suitable material, e.g., polymer, dielectric, semiconducting, non-magnetic metal, or photoresist material. Photoresist material can be a positive or a negative photoresist for photo-lithography. The supporting material 122 can be configured to form the photonic structure 120 on a surface of the supporting material 122, e.g., as described with further details in FIG. 5. In some examples, the supporting material 122 is SU-8.

The photonic structure 120 can be formed on a surface of the supporting material 122. The magnetic structure can be formed within the supporting material 122, e.g., at a center of the supporting material 122.

### Example Photonic Structures

FIG. 2A illustrates various examples of magneto-photonic particles for light delivery and collection with corresponding photonic structures.

For example, diagram (a) of FIG. 2A shows a magneto-photonic particle 200a configured for light delivery, which includes a supporting material 202a (e.g., the supporting material 122 of FIG. 1), a magnetic structure 206a, and a photonic structure 204a (e.g., the photonic structure 120 of FIG. 1). Incident light 201a can encounter the photonic structure 204a, which can be configured to redirect (e.g., diffract, scatter, or reflect) the incident light towards a target 210a in a medium as redirected light 203.

The incident light 201a can be from a light source (e.g., the light source 104 of FIG. 1) that can be a laser or different light source. The light source can be external to the medium that contains the magneto-photonic particle 200a and target 210. Both the location and orientation of the magneto-photonic particle 200a relative to the light source of the incident light 201a can be controlled such that the photonic structure 204a delivers light to the target 210a. Additionally, the characteristics of the photonic structure 204a can be selected to redirect incident light 201a toward the target 210a. Further, the location and orientation of the photonic structure 204a can be controlled to redirect the incident light 201a toward the target 210a, e.g., by controlling the magnetic structure in the magneto-photonic particle 200a as discussed herein. In some embodiments, the photonic structure 204a can be designed using one or more wavelengths of the incident light 201a, e.g., as illustrated with further details in FIG. 2B.

Diagram (b) of FIG. 2A shows another example magneto-photonic particle 200b for light delivery. The magneto-photonic particle 200b can be configured to function as a focusing reflector (e.g., mirror). The magneto-photonic particle 200b can include a supporting material 202b (e.g., the supporting material 122 of FIG. 1), a magnetic structure 206b, and a photonic structure 204b (e.g., the photonic structure 120 of FIG. 1). Light 201b illuminates the photonic structure 204b, and the photonic structure 204b can be configured to focus substantially parallel light 201b at a particular distance, e.g., a distance to the target 210b, away from the photonic structure 204b, and the focused light 203b can be incident on the target 210b.

Diagram (c) of FIG. 2A shows another example magneto-photonic particle 200c configured for light collection. The magneto-photonic particle 200c can include a support material 202c (e.g., the supporting material 122 of FIG. 1), a magnetic structure 206c, and a photonic structure 204c. A target 210c can emit light, e.g., luminescent or fluorescent light, reflect light, or scatter light. Light 201c from the target 210c can encounter the photonic structure 204c that can be configured to redirect (e.g., diffract, scatter, or reflect) the emitted light 201c in a particular direction as redirected light 203c, e.g., away from the magneto-photonic particle 200c and towards a light detector (e.g., the light detector 108 of FIG. 1).

Diagram (d) of FIG. 2A shows another example magneto-photonic particle 200d configured for light collection. The magneto-photonic particle 200d can include a supporting material 202d (e.g., the supporting material 122 of FIG. 1), a magnetic structure 206d, and a photonic structure 204d. A target 210d to be detected or imaged can emit, reflect, or scatter light towards the photonic structure 204d. The photonic structure 204d can be configured to redirect (e.g., diffract or reflect) the light 201d from the target 210d at a distance away from the magneto-photonic particle 200d, e.g., towards a light detector (e.g., the light detector 108 of FIG. 1). Redirected light 203d can be parallel and can then be focused onto the light detector.

Diagram (e) of FIG. 2A shows another example magneto-photonic particle 200e configured to both direct and collect light, which includes a supporting material 202e (e.g., the supporting material 122 of FIG. 1), a magnetic structure 206e, and a photonic structure 204e. In this example, incident light 201e1, e.g., light from a laser, can encounter the photonic structure 204e. The photonic structure 204e can be configured to redirect the incident light 201e1 as redirected light 203e1 toward a target 210e. In response to receiving the redirected light 203e1, the target 210e can reflect, scatter, or absorb and then emit light. Light 201e2 from the target 210e can encounter the photonic structure 204e again that can be configured to redirect the light 201e2 as redirected light 203e2 along a particular direction.

In some embodiments, the photonic structure 204e can have wavelength dependent properties. For example, incident light 201e1 can have a first wavelength λ₁, and light 201e2 can have a second wavelength λ₂. The photonic structure 204e can be configured to direct light of first wavelength λ₁ towards the target 201e and light of the second wavelength λ₂ towards a light detector such as a camera.

FIG. 2B illustrates examples of magneto-photonic particles for light manipulation with corresponding wavelength dependent photonic structures. For example, diagram (a) of FIG. 2B shows an example magneto-photonic particle 250a configured to deflect, e.g., diffract, light at different angles depending on the wavelengths. The magneto-photonic particle 250a can include a supporting material 252a, a magnetic structure 256a, and a photonic structure 254a. The photonic structure 254a can be configured to direct incident light 251a1 of a first wavelength λ₁ along a first direction and incident light 251a2 of the second wavelength λ₂ along a second direction. Consequently, redirected light 253al and 253a2 can propagate in the first and second directions, respectively.

Diagram (b) of FIG. 2B shows another example magneto-photonic particle 250b configured to deflect light of a first wavelength λ₁ and absorb light of a second wavelength λ₂. The magneto-photonic particle 250b can include a supporting material 252b, a magnetic structure 256b, and a photonic structure 254b. Incident light 251b1 of the first wavelength λ₁ can encounter the photonic structure 254b and be deflected as redirected light 253b. Incident light 251b2 of the second wavelength λ₂ can encounter the photonic structure 254b and be absorbed by the magneto-photonic particle 250b.

Diagram (c) of FIG. 2B shows another example magneto-photonic particle 250c configured to deflect light of a first wavelength λ₁ and transmit light of a second wavelength λ₂. The magneto-photonic particle 250c can include a supporting material 252c, a magnetic structure 256c, and a photonic structure 254c. Incident light 251c1 of the first wavelength λ₁ can encounter the photonic structure 254c and be deflected as redirected light 253c. Incident light 251c2 of the second wavelength λ₂ can encounter the photonic structure 254c and be transmitted through the magneto-photonic particle as transmitted light 255.

In some embodiments, the material choices of the photonic structure 254c, supporting material 252c, and magnetic structure 256c can be chosen to transmit light within a particular frequency range. The shape of the magnetic structure 256c can be chosen to allow transmission of light within the particular frequency range. For example, instead of the solid core, the magnetic structure 256c can be engineered to include microstructures or one or more thin layers (nanoscale slivers), arranged throughout the supporting material 252c to allow transmission of light within the particular frequency range.

Although the magneto-photonic particles illustrated in FIGS. 2A-2B can have different photonic structures for different applications, the magnetic structures in the magneto-photonic particles can be same or similar, and be configured to be directionally controllable, e.g., as discussed with further details below.

### Example Magnetic Structures

FIG. 3 illustrates an example magnetic structure 304 in a particle 300. The particle 300 can be an example of the magneto-photonic particle 102 of FIG. 1 before a photonic structure is formed on the top surface. The particle 300 can be a circular disk, e.g., have a circular outline when viewed from above as in the “top view” of FIG. 3. A magnetic structure 304 can be embedded within a supporting material 302, e.g., the surface of the particle 300 does not include a portion corresponding to the magnetic structure 304. In general, the shape of magnetic structure 204 can lack a circular symmetry, such that it can be oriented along a preferred direction by a magnetic field, e.g., be directionally orientable.

In this example, the particle 300 has a first width w₁ corresponding to the diameter of the particle 300. The first width w₁ of the particle 300 can be in a suitable range, e.g., 1 to 100 μm such as 50 to 100 μm. In some examples, the magnetic structure 304 can be a triangular prism, e.g., have a triangular outline cross section when viewed from the “top view.” The triangular outline can correspond to an isosceles triangle, the triangle having a width w₂ and a height h₁. The width w₂ can be smaller than the first width w₁, e.g., 40 μm. The height h₁ can be smaller than the first width w₁ of the particle 300, e.g., 70 μm. The main constraint is that the size of the magnetic structure be no larger than that of the overall structure of the particle 300.

A “side view,” e.g., a cross-section of the particle 300, can reveal a rectangular shape of both the particle 300 and the magnetic structure 304. The magnetic structure 304 can be embedded within the supporting material 302 (e.g., in a center of the supporting material 302), such that the top and bottom of the magnetic structure 304 do not reach the top 306 and/or bottom 308 of the particle 300. In some examples, a height h₂ of the supporting material 302 below the magnetic structure 304 can be in a suitable range, e.g., 100 nm to 1 μm such as 500 nm. A height h₃ of the magnetic structure 304 can be in a suitable range, e.g., 10 nm to 100 nm such as 50 nm. The height h₄ of the supporting material 302 above the magnetic structure 304 can be identical to or larger than the height h₂ of the supporting material 302 below the magnetic structure 304. In some embodiments, the heights of the supporting material 302 above and below the magnetic structure 304 being substantially the same can facilitate a uniform control of the orientation of the particle 300. For example, the height h₄ can be in a suitable range, e.g., hundreds of nm to several μm, such as 1 μm.

The supporting material 302 can be transparent, such that the magnetic structure 304 is visible from a top and bottom view of the particle 300 before a photonic structure is formed on either side.

In some embodiments, the magnetic structure 304 does makeup a portion of the bottom 308 of the particle 300, e.g., the height h₂ between the bottom 308 of the magneto-photonic particle 300 and the bottom of the magnetic structure 304 is zero.

FIG. 4 shows example controllable movements of a particle 402 with a non-symmetric magnetic structure 403. The particle 402 can be an example of the magneto-photonic particle 102 of FIG. 1 or the particle 300 of FIG. 3. A magnetic source 406 (e.g., the magnetic source 106 of FIG. 1) can generate a magnetic field 401 that the particle 402 experiences. For example, the magnetic field 401 can extend through the particle 402, causing the particle 402 to translate in space relative to the magnetic source 406 and change orientation, due to the magnetic structure 403 lacking circular symmetry. The magnetic source 406 can be an external magnet.

The effect of the magnetic field on the particle 402 can differ based on the distance between the magnetic source 406 and the particle 402. For example, when the magnet source 406 is far enough away from the particle 402 (e.g., greater than 10 cm such as 20 cm), the magnetic source 406 can primarily cause rotation of the particle 402. This distance can depend on the strength of the magnet source 406. Scenes 411, 413, and 415 depict the particles 402 changing their orientation in response to the magnetic source 406 moving, e.g., rotating or moving relative to the particles 402. In some examples, the magnetic source 406 can be moved along a circular path to induce rotation of a particle 402. The circular path can be approximately concentric with the position of the particle 402.

In scene 411, the magnetic source 406 has a first orientation 405, e.g., “up.” This first orientation 405 causes the magnetic source 406 to generate a magnetic field, which causes the triangular magnetic structure 403 to point along the direction of the first orientation 405.

In scene 413, the orientation of the magnetic source 406 changes to a second orientation 407, e.g., “left.” The second orientation 407 causes the magnetic source 406 to generate a magnetic field, which causes the triangular magnetic structure 403 to point along the direction of the second orientation 407.

In scene 415, the orientation of the magnetic source changes to a third orientation 409, e.g., “right.” The third orientation 409 causes the magnetic source 406 to generate a magnetic field, which causes the triangular magnetic structure 403 to point along the direction of the third orientation 409.

Because the magnetic source 406 primarily affects the orientation of the particles 402 in the far field limit, the location in space of the particles 402 does not notably change when the orientation of the magnetic source 406 does. Because this particular particle 402 has a disk shape, e.g., a cylinder, it can rotate about an axis through the center of the cylinder without changing its location relative to the magnetic source 406.

As another example, when the magnetic source 406 is close enough to the particle (e.g., less than 10 cm), the magnetic source 406 can primarily cause translation of the particle 402. This distance can depend on the strength of the magnetic source 406. Scenes 410, 420, 422, and 426 depict a series of translations of the particle 402 in response to the magnetic source 406 moving. In some examples, the magnetic source 406 is moved along a same line designed for the particle 402 to move. The magnetic source 406 can be translated, without rotation.

Scene 410 depicts a particle 402, a target, and a series of three steps to arrive at the target. The particle 402 starts at a first position 412. The location and orientation of the magnetic source 406 cause the magnetic source 406 to generate a magnetic field that causes the particle 402 to translate along a first path 414. Along the first path 414, the orientation of the magnetic structure 403 does not notably change, e.g., the triangular outline of the magnetic structure 403 remains pointing down.

In some examples, e.g., as shown in scenes 420, 422, 426 below, the particle 402 is both oriented and translated to be guided to reach a desired position at an end of a second path. The triangular core of the magnetic structure 403 can be aligned with the direction of the translation.

Scene 420 depicts the particle 402 after it has translated to a second position 416 by moving along the first path 414. Moving the magnetic source 406, e.g., translating, rotating, or both relative to the particle 402 can cause translation along a second path 418.

Scene 422 depicts the particle 402 after it has translated to a third position 425. The orientation of the particle 402 changes between scenes 420 and 422. Moving the magnetic source 406, e.g., translating, rotating, or both, relative to the particle 402 can cause translation along a third path 424.

Scene 426 depicts the particle 402 having arrived at the target location 428 by moving along the third path 424. Once again, the orientation of the particle 402 can change between scenes 426 and 422.

### Example Fabrication Process

FIG. 5 shows an example process 500 of fabricating magneto-photonic particles 501. The magneto-photonic particles 501 can be the particles 102 of FIG. 1, 200a-200e of FIG. 2A, 254a-254c of FIG. 2B, 300 of FIG. 3, or 402 of FIG. 4. In general, atomic layer deposition (ALD), nano-imprinting, lithography methods and systems, or any other suitable methods and systems can be used to perform the process 500.

The process 500 can start with providing a substrate 502. The substrate 502 can be, for example, a silicon wafer. A first polymer 504 can be deposited onto the substrate 502. In some embodiments, the first polymer 504 is spin coated to provide a uniform coating on top of the substrate 502. In some embodiments, the first polymer 504 is an epoxy-based, negative photoresist, such as SU-8.

The first polymer 504 can be patterned to form multiple discrete supporters 506 on the substrate 502. For example, the pattern can correspond to evenly-spaced circular disks, such that the portion of the first polymer 504 not corresponding to the circular disks is removed from the substrate 502. In some examples, a supporter 506 includes cross-linked SU-8. For example, SU-8 film can be prepared as the first polymer 504 on the substrate by spin coating and pre-annealing process. Then, the SU-8 film can be exposed region-selectively by ultraviolet (UV) light using photolithography, which can create radicals in exposed regions. Then, post-annealing process can make the radicals active and create the SU-8 cross-linked.

For each supporter 506, a respective magnetic structure 508 can be formed on the surface of the discrete supporters 506. In some embodiments, the magnetic structure 508 is formed by a metal lift-off method. In some embodiments, the magnetic structure 508 is cobalt. The material choice of the magnetic structure 508 can allow for the magnetic structure 508 to be controllable by a magnetic field while remaining in place while other layers dissolve during fabrication, e.g., compatible with the material of the supporters 506.

The substrate 502 with the supporters 506 on top can receive a second polymer 510, which can completely cover the supporters 506. The second polymer 510 can include a supporting material, e.g., the material that supports the magnetic structure 508 and holds it in place relative to the first polymer 504 layer. The second polymer 510 can be made of the same material of the first polymer 504, e.g., SU-8.

The second polymer 510 can form a separation layer on top of the magnetic structures 508, e.g., a portion of the second polymer 510 can later be dissolved to release the magneto-photonic particles 501.

In some embodiments, the second polymer 510 is an uncured, epoxy-based photoresist, such as uncured SU-8. In some embodiments, the first polymer 504, the second polymer 510, or both are transparent, such that the supporters 506 with their respective magnetic structures 508 are visible when viewed from above. In some embodiments, the first polymer 504, the second polymer 510, or both are a nonmagnetic material.

Because the second polymer 510 is uncured, the second polymer 510 can be nano-imprinted with a stamp 514 to form a photonic structure 516 on the surface of the second polymer 510. The stamp 514 can have preconfigured microstructure and/or nanostructures. In some embodiments, the photonic structure 516 is formed using e-beam lithography. The photonic structure 516 can include micro- and/or nanoscale photonic features. The photonic structure 516 can be the photonic structure 120 of FIG. 1, any one of 204a-204e of FIG. 2A, or any one of 250a-250c of FIG. 2B.

The second polymer 510 can be patterned such that the discrete circular disks, e.g., the supporters 506, remain on the substrate 502. The pattern used to pattern the first polymer 504 can be the same as that used to pattern the second polymer 510. As a result of the patterning, each supporter 506 has a photonic structure 512, which corresponds to a portion of the photonic structure 516.

The supporters 506 can be released from the substrate 502 to form multiple magneto-photonic particles 501, each having a photonic structure 512 and a magnetic structure 508. In some limitations, releasing the supporters 506 from the substrate 502 can include a sacrificial layer dissolution, e.g., dissolving at least part or all of the substrate 502.

Optionally, before releasing the supporters 506 from the substrate 502, a layer of metal 520 can be deposited onto the supporters 506 and substrate 502. As a result, the magneto-photonic particles 501 have a metallic coating on top of the photonic structure 512. A metallic coating can be beneficial, because, although the photonic structure 512 might have a desirable morphology, it might not be highly efficient in reflecting light. For example, the contrast in the refractive indices between air and SU-8 is less than the contrast in refractive indices between air and metal, which can lead to a low reflection coefficient of the photonic structure 512. In some embodiments, a silicon nitride/silicon dioxide (Si₃N₄/SiO₂) dielectric material can be used in place of metal as a coating for the photonic structure 512.

### Example Applications

FIG. 6A shows an example fabricated magneto-photonic particle 600 (e.g., the magneto-photonic particle 501 fabricated by the process 500 of FIG. 5). A front view 602 shows the top of the magneto-photonic particle 600, e.g., the photonic surface with a metal coating. A back view 604 shows the bottom of the magneto-photonic particle 600. Because the supporting material between the magnetic coating and the bottom of the magneto-photonic particle 600 is transparent, the triangular magnetic structure is visible in the back view 604. The magneto-photonic particles 600 can be, for example, the magneto-photonic particles 102 of FIG. 1, 200a-200e of FIG. 2A, 254a-254c of FIG. 2B, 300 of FIG. 3, or 402 of FIG. 4.

FIG. 6B shows an example system 621 for controlling the magneto-photonic particle 600 of FIG. 6A. The system 621 can be similar to, or the same as, the system 100 of FIG. 1. The system 621 can include a light source 606, a hemispherical dome 610 filled with the medium 628 such as water, a platform 612 that supports the hemispherical dome 610, and magneto-photonic particles 600 immersed in the medium 628. In this example, the magneto-photonic particles 600 can be configured to diffract light of a particular wavelength, e.g., 635 nm. The light source 606 can shine a laser beam 608 into an opening of the hemispherical dome 610 at a particular polar coordinate 614, e.g., 0° as depicted in FIG. 6B.

Depending on the orientation of the magneto-photonic particles 600, e.g., the direction in which the triangular magnetic structure points (measured by the azimuthal coordinate 616), the diffraction pattern (e.g., diffracted light at a series of diffraction orders) seen in the top view 618 can differ its orientation.

FIG. 6C shows results of controlling light steering directions of the magneto-photonic particle 600 of FIG. 6A by controlling orientations of the magneto-photonic particle 600. The magnetic source can move, e.g., translate or rotate relative to the magneto-photonic particle 600, to cause the orientation of the magneto-photonic particle 600 to change, as discussed in relation to FIG. 4. The hemispherical dome 610 can be translucent, such that light diffracted by the magneto-photonic particles 600 within the medium 628 can illuminate the hemispherical dome 610.

In the first scene 601, the orientation 622 of the magneto-photonic particle 600 is slightly tilted to the right, and the resulting diffraction pattern 620 is also slightly tilted to the right. In the second scene 603, the orientation 626 of the magneto-photonic particle 600 is upright, and the resulting diffraction pattern 624 is also upright. In the third scene 605, the orientation 630 of the magneto-photonic particle 600 slightly tilted to the left, and the resulting diffraction pattern 632 is also slightly tilted to the left. Accordingly, the direction of light delivery can be controlled by orienting magneto-photonic particle 600.

### Example Processes

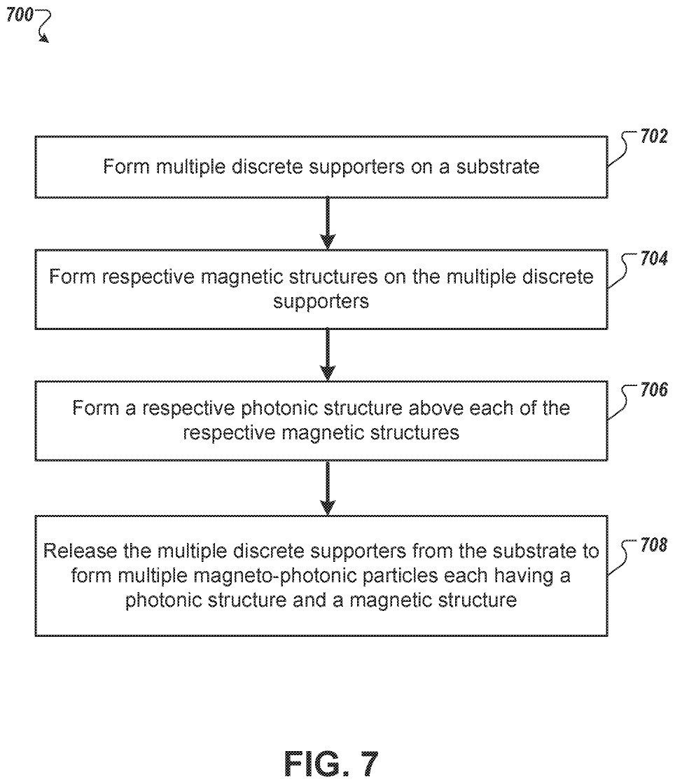

FIG. 7 is a flowchart of an example process 700 for forming magneto-photonic particles. The magneto-photonic particles can be any of the magneto-photonic particles 102, 200a-200e, 250a-250c, 300, 402, 501, and 600, as discussed in the present disclosure. The process 700 can be similar to, or the same as, the process 500 of FIG. 5.

A deposition system can form multiple discrete supporters on a substrate (702). In some embodiments, forming the multiple discrete supporters (e.g., the supporters 506 of FIG. 5) includes depositing a polymer (e.g., the first polymer 504 of FIG. 5) on the substrate and patterning the polymer according to a pattern corresponding to the multiple discrete supporters, e.g., multiple circular disks.

The deposition system can form respective magnetic structures on the multiple discrete supporters (704). In some embodiments, forming the respective magnetic structures (e.g., the magnetic structures 508 of FIG. 5) on the multiple discrete supporters includes depositing a magnetic material (e.g., a magnetic metal such as cobalt) on the plurality of discrete supporters; and performing, on each discrete supporter, a lift-off of a portion of the magnetic material.

In some embodiments, the respective magnetic structures include a magnetic material that is compatible with a material of the multiple discrete supporters.

The deposition system can form a respective photonic structure above each of the respective magnetic structures (706). In some embodiments, forming the respective photonic structure (e.g., the photonic structure 516 of FIG. 5) above each of the respective magnetic structures includes: imprinting a separation layer (e.g., the second polymer 510 of FIG. 5) with a photonic pattern on a stamp (e.g., stamp 514 of FIG. 5); depositing a metal layer on the imprinted pattern; or both.

The deposition system can release the multiple discrete supporters from the substrate to form multiple magneto-photonic particles each having a photonic structure and a magnetic structure (708). In some embodiments, releasing the multiple discrete supporters from the substrate to form the multiple magneto-photonic particles includes dissolving the substrate to release the multiple discrete supporters.

In some embodiments, the process 700 can include one or more additional steps, fewer steps, or some of the steps can be divided into multiple steps. As an example, the process 700 can include forming a separation layer on top of the respective magnetic structures. As another example, after step 704, a deposition system can deposit a layer of a polymer, portions of which can later be dissolved as a sacrificial layer.

In some embodiments, the multiple discrete supporters and the separation layer include the same material, e.g., an epoxy-based, negative photoresist such as SU-8. In some embodiments, the multiple discrete supporters and the separation layer include different materials.

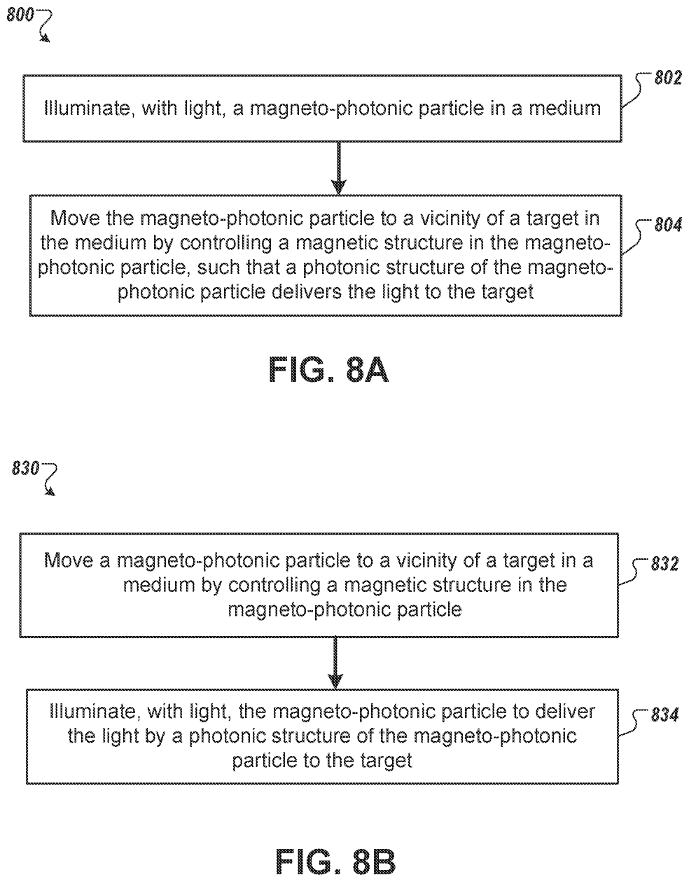

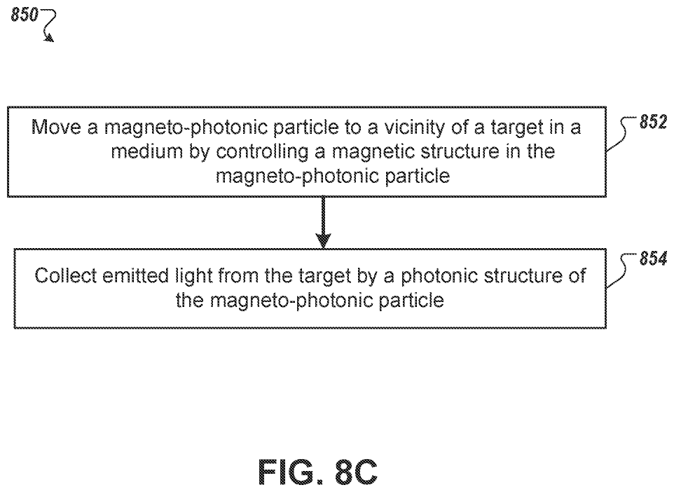

FIG. 8A is a flow chart of an example process 800 for operating magneto-photonic particles for delivering light. The process 800 can be performed by a system, e.g., the system 100 of FIG. 1 or the system 621 of FIG. 6B. The magneto-photonic particles can be any of the magneto-photonic particles 102, 200a, 200b, 200e, 250a-250c, 300, 402, 501, and 600.

The system can illuminate, with light, a magneto-photonic particle in a medium (802). In some embodiments, one or more magneto-photonic particles can be immersed in the medium. The medium can include a biological or biomedical medium.

The system can move the magneto-photonic particle to a vicinity of a target in the medium by controlling a magnetic structure in the magneto-photonic particle, such that a photonic structure of the magneto-photonic particle delivers the light to the target (804). The target can include a biological or biomedical object such as a molecule, a cell, or a tissue.

In some embodiments, the process 800 can include one or more additional steps, fewer steps, or some of the steps can be divided into multiple steps. For example, the process 800 can include orienting the magneto-photonic particle to direct the light by the photonic structure to the target.

In some limitations, the process 800 includes collecting light coming from the target by the photonic structure of the magneto-photonic particle. The light coming from the target can include at least one of: scattered light, reflected light, or emitted light, e.g., the target can be a fluorescent molecule.

FIG. 8B is a flow chart of another example process 830 for operating magneto-photonic particles for delivering light. The process 830 can be performed by a system, e.g., the system 100 of FIG. 1 or the system 621 of FIG. 6B. The magneto-photonic particles can be any of the magneto-photonic particles 102, 200a, 200b, 200e, 250a-250c, 300, 402, 501, and 600. Different from the process 800 of FIG. 8A, the process 830 can be performed by first moving the magneto-photonic particles before illuminating light.

The system can first move a magneto-photonic particle to a vicinity of a target in a medium by controlling a magnetic structure in the magneto-photonic particle (832). In some embodiments, moving the magneto-photonic particle to the vicinity of the target in a medium can include translating or rotating a magnetic controller relative to the magneto-photonic particle. In some embodiments, translating or rotating the magnetic controller relative to the magneto-photonic particle causes the magneto-photonic particle to translate or rotate relative to the magnet.

After the magneto-photonic particle is moved to the vicinity of the target in the medium, the system can illuminate, with light, the magneto-photonic particle to deliver the light by a photonic structure of the magneto-photonic particle to the target (834).

FIG. 8C is a flow chart of another example process 850 for operating magneto-photonic particles for collecting light. The process 830 can be performed by a system, e.g., the system 100 of FIG. 1 or the system 621 of FIG. 6B. The magneto-photonic particles can be any of the magneto-photonic particles 102, 200c, 200d, 200e, 250a-250c, 300, 402, 501, and 600.

The system can move a magneto-photonic particle to a vicinity of a target in a medium by controlling a magnetic structure in the magneto-photonic particle (852). The system can then collect emitted light from the target by a photonic structure of the magneto-photonic particle (854).

The order of steps in the processes 800, 830, and 850 described above is illustrative only, and forming magneto-photonic particles can be performed in different orders. For example, the location of the target can change, and the system can repeatedly illuminate and move the magneto-photonic particle throughout the medium. Processes 800, 830, and 850 can be combined for applications involving both the collection and delivery of light via the magneto-photonic particle.

While this specification contains many specific implementation details, these should not be construed as limitations on the scope of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

Further modifications and alternative embodiments of various aspects will be apparent to those skilled in the art in view of this description. Accordingly, this description is to be construed as illustrative only. It is to be understood that the forms shown and described herein are to be taken as examples of embodiments. Elements and materials may be substituted for those illustrated and described herein, parts and processes may be reversed, and certain features may be utilized independently, all as would be apparent to one skilled in the art after having the benefit of this description.

What is claimed is:

1. A particle comprising:
a supporting material;
a photonic structure configured to manipulate light; and
a magnetic structure controllable to make the particle move,
wherein the photonic structure and the magnetic structure are supported by the supporting material.

2. The particle of claim 1, wherein the photonic structure is configured to diffract, reflect, focus, scatter, steer, direct, absorb, or transmit the light.

3. The particle of claim 1, wherein the photonic structure is configured to deliver the light towards a target in a medium.

4. (canceled)

5. The particle of claim 1, wherein the photonic structure is configured to collect the light from a target in a medium.

6. (canceled)

7. The particle of claim 1, wherein the photonic structure is configured to diffract, steer, scatter, direct, or focus light with a first wavelength towards a first direction and light with a second wavelength towards a second direction.

8. The particle of claim 1, wherein the photonic structure is configured to diffract, steer, scatter, direct, or focus light with a first wavelength and absorb light with a second wavelength.

9. (canceled)

10. (canceled)

11. The particle of claim 1, wherein the photonic structure comprises a diffractive grating, a meta grating, a metasurface, or a phase-gradient metasurface.

12. The particle of claim 1, wherein the magnetic structure is configured to be directionally orientable and movable towards a target in the medium.

13. (canceled)

14. (canceled)

15. (canceled)

16. The particle of claim 1, wherein the magnetic structure comprises an engineered structure of a magnetic material, the engineered structure configured to transmit the light from the photonic structure.

17. The particle of claim 1, wherein the magnetic structure comprises at least one of cobalt, nickel, iron, iron-oxide, or an alloy comprising at least one magnetic material.

18. (canceled)

19. The particle of claim 1, wherein the supporting material comprises at least one of a polymer material, a dielectric material, a semiconducting material, a photoresist material, or a non-magnetic metal.

20. The particle of claim 1, wherein the particle has a disc shape with a diameter at an order of ones to hundreds of micrometers.

21. (canceled)

22. (canceled)

23. (canceled)

24. The particle of claim 1, wherein the supporting material comprises a top surface and a bottom surface along a direction, and
wherein the photonic structure is formed on the top surface of the supporting material, and the magnetic structure is embedded in the supporting material.

25. The particle of claim 24, wherein a thickness of the supporting material from the top surface to the bottom surface along the direction is in an order of hundreds of nanometers to tens of micrometers.

26. The particle of claim 25, wherein the magnetic structure has an upper edge and lower edge and has a thickness from the upper edge to the lower edge along the direction in an order of ones to tens of nanometers.

27. (canceled)

28. (canceled)

29. The particle of claim 26, wherein the upper edge and lower edge of the magnetic structure each have a substantially isosceles triangular shape in a plane perpendicular to the direction, and
wherein a width and a height of the triangular shape are at an order of tens of micrometers.

30. The particle of claim 1, wherein an outer surface of the particle comprises one or more functional chemical or biological groups.

31.-35. (canceled)

36. A method of forming magneto-photonic particles, comprising:
forming a plurality of discrete supporters on a substrate;
forming respective magnetic structures on the plurality of discrete supporters;
forming a respective photonic structure above each of the respective magnetic structures; and
releasing the plurality of discrete supporters from the substrate to form a plurality of magneto-photonic particles each having a photonic structure and a magnetic structure.

37.-54. (canceled)

55. A system comprising:
one or more magneto-photonic particles; and
a magnetic controller configured to directionally orient or move the one or more magneto-photonic particles,
wherein each of the one or more magneto-photonic particles comprises:
a supporting material;
a photonic structure configured to manipulate light; and
a magnetic structure controllable by the magnetic controller to make the magneto-photonic particle move,
wherein the photonic structure and the magnetic structure are supported by the supporting material.

56. (canceled)

57. (canceled)

58. The particle of claim 1, wherein the photonic structure comprises at least one of a dielectric material, a high refractive index material, or a non-magnetic material.
